(19) United States
(12) Patent Application Publication
(10) Pub. No.: US 2021/0230521 A1
(43) Pub. Date: Jul. 29, 2021

(54) METHOD FOR PRODUCING WINE WITH A LOW ALCOHOL CONTENT, AND FERMENTING-DESUGARING UNIT FOR THE IMPLEMENTATION THEREOF

(71) Applicant: AB7 INDUSTRIES S.A, Deyme (FR)

(72) Inventor: René CHELLE, Deyme (FR)

(21) Appl. No.: 17/168,323

(22) Filed: Feb. 5, 2021

Related U.S. Application Data
(63) Continuation of application No. 16/310,182, filed on Dec. 14, 2018, now abandoned.

(30) Foreign Application Priority Data
Jun. 16, 2016 (FR) 1600961

(51) Int. Cl. C12G 1/022; C12M 1/00

(52) U.S. Cl. CPC C12G 1/0203; C12M 21/00

(57) ABSTRACT

According to the invention, an aliquot of must is sent from a wine making vat into an air-lift-type fermenting-desugaring unit, wherein homogenization of the aliquot and aeration of the yeasts are carried out with controlled heating in order to achieve aerobic fermentation until all the sugar in the aliquot has been consumed completely.

The present invention relates to the fermentation of alcoholic beverages from a grape juice. It relates to a process for the preparation of a wine with a reduced content of alcohol using only biological methods of fermentation of natural fruit juice. This method allows in particular to produce, from a must whose alcoholic potential P is commonly from 8° to 15°, a beverage having an alcohol content at least 4° lower than its alcoholic potential P. The invention also relates to a device for implementing the aforementioned method.

We know that the fight against alcoholism is an important public health issue. However, non-alcoholic wines, which have been proposed to date, have not been favorably received by consumers because of the fact that they have poorly developed taste characteristics. These drinks are often obtained by fermentation of all the sugars present in the grape juice, and then extraction of a portion of the alcohol formed by physical methods (see, in particular patents EP-A 0193206 or EP-A 228572); in this case, their production therefore requires at least one additional step, which corresponds to an increase in cost. In addition, it appears that this extraction eliminates or degrades most of the compounds responsible for the organoleptic properties and qualities of the wine.

The present invention provides a method using only biological fermentation methods by using a yeast culture; this method is based on fermentation mechanisms identical to those commonly used for the production of wines. The resulting wine has attractive organoleptic qualities for the consumer.

The present invention provides a method using only biological fermentation methods by using a yeast culture; this method is based on fermentation mechanisms identical to those commonly used for the production of wines. The resulting wine has attractive organoleptic qualities for the consumer.

Some producers have already proposed a method (see EP 0440975) comprising different steps under more or less controlled continuous aeration to obtain a wine with reduced alcohol content. In this method, it is proposed to carry out a first alcoholic fermentation step of a fraction of grape juice under limited supply of oxygen, this leading to the production of alcohol; this step is then followed by an aerobic process after the addition of a second fraction of juice. In this method, the alcohol produced in the first step becomes a substrate and is, in fact consumed by the yeasts during the second step, which considerably reduces the yield of the whole. In addition, the renewal of the fermenting yeast population is no longer ensured after total sugar consumption during the second step. Therefore, this method is not satisfactory.

In the French patent 2887257, the method proposed therein essentially comprises the steps of:

a. feeding an aerobic culture reactor containing yeasts in the multiplication phase, with a first fraction of grape juice;

b. feeding an anaerobic fermentation reactor with the yeast-laden medium obtained in step a and a second fraction of grape juice to obtain a must;

c. filter the must to separate the yeasts and the fermented beverage obtained. In a variant, the sugar of the first fraction is completely or almost completely consumed in the culture reactor. In this method, the first culture reactor causes the multiplication of the yeasts that are introduced and the first reactor feeds the second reactor, namely, a fermentation reactor, which operates in anaerobic manner, so that the yeasts consume their nutrients and especially the alcohol and sugar found in the medium contained in the first reactor; in the second reactor, the large quantity of yeasts allows to obtain a low level of alcohol; and, in this way, the outlet of the second reactor being filtered to separate the yeasts, it constitutes the fermented beverage with a desired low content of alcohol.

There is therefore a need to provide a simplified device for the continuous production of a wine of low alcohol content with good organoleptic qualities by means of a single specific reactor capable of removing the sugar from the must thanks to the yeasts whose proliferation within said reactor is optimized. Similarly, there is a need to provide a method whose implementation can gradually dilute the sugar content of an initial must to be processed while ensuring a fast restart of the wine making process.

According to the present invention, it was found that it was possible to significantly simplify the device proposed by EP 1891199 patent while retaining the principle according to which the sugar-removing process of the must is carried out on a processed fraction which is then remixed with the unprocessed fraction left in the wine making vat. Moreover, the method according to the invention allows, in one variant, to obtain a large quantity of yeasts that can be used for seeding vats and for improving the organoleptic qualities of wine with a low alcohol content; it is known, indeed, that the taste of wine can be improved by weakening the cell walls of the yeasts, which leads to the provision of polysaccharides or various aromatic compounds, which contribute as substrates, for example, to the start of the malolactic fermentation. By implementing the invention, “fresh” yeasts produced by the method according to the invention can be valorized, whereas, to date, wine producers have been purchasing dehydrated yeasts whose conservation is limited in time (about 5 to 8 weeks) and which, moreover, are sold in the form of powder mixed with various cellular debris.

The subject-matter of the present invention is, therefore, a method for preparing a wine having good organoleptic qualities and a low alcohol content d from a grape must, whose high sugar content would lead, after a conventional fermentation process, to a wine of potential low alcohol content D>d, said must being in a wine making vat from which an aliquot of must is taken in a fermentation system using yeasts, said system having two fermentation steps, one of which is aerobic and the other anaerobic, the output flow of the first step feeding the second step, which provides the processed aliquot and returns it to the wine making vat to be mixed therein with the initial remaining must, characterized in that, in a single fermenting-desugaring unit, the homogenization of the fermentation medium and the aeration of the yeasts is ensured simultaneously, the multiplication in aerobic mode of the yeasts consuming sugar without producing ethanol, by means of a flow of air associated with a circulating flow of the aliquot from the wine making vat, said homogenization taking place with a controlled heating which brings the temperature of the aliquot to a value corresponding to a suitable fermentation in aerobic mode until a complete or almost complete consumption of the sugar of the aliquot of the processed must.

In a preferred embodiment of the method defined above, the circulating flow of the aliquot of must being processed and the associated air circulation are carried out from bottom to top, parallel to the average line of the fermenting-desugaring unit. According to this variant, the fermenting-desugaring unit operates according to a known mode, which is generally called “air lift.”

In one embodiment of the method according to the invention, the heating of the fermenting-desugaring unit maintains, in the fraction of the must being processed that it contains, a substantially homogeneous temperature of between 30 and 45° C. and, preferably, close to 40° C.

According to an embodiment of the method according to the invention, the aliquot of must devoid of sugar reintroduced in the wine making vat contains yeasts, and comes from the fermenting-desugaring unit, in order to seed the remaining must in said vat to ensure a faster restart of the wine making process.

According to an embodiment of the method according to the invention, it is possible to use the heating of the fermenting-desugaring unit in a cleaning or disinfecting phase of the fermenting-desugaring unit to bring to a boil a quantity of wash water, which, after condensation, is evacuated through a drain valve.

According to an embodiment of the method according to the invention, the sugar content of the aliquot of must being processed, which circulates in the fermenting-desugaring unit, is continuously measured by means of a refractometer to regulate automatically, by activating the air supply, the aerobic mode of fermentation.

According to an embodiment of the method according to the invention, the measurement of the sugar level of the aliquot being processed in the fermenting-desugaring unit is used to determine the moment when all the sugar of said fraction has been consumed by the aerobic fermentation and then order the immediate shutdown of the air inflow and heating in the fermenting-desugaring unit, to avoid any deterioration of the organoleptic qualities of said aliquot.

According to an embodiment of the method according to the invention, the microbial density, and thus the growth of the yeasts, is measured in the fraction being processed by means of a photometer and the fermentation is thus controlled in a complementary manner in the fermenting-desugaring unit.

According to another embodiment of the method according to the invention, the aliquot devoid of sugar being processed is transferred from the fermenting-desugaring unit to a settling vat equipped with a cooling means, to obtain the desired clarification of the must. In this embodiment of the method, all or part of the yeasts which have sedimented in the form of a dense yeast-concentrated cream are collected in the settling vat and the said yeasts are reintroduced into the wine making vat to ensure, at least partially, seeding of said vat. According to another embodiment, the organoleptic qualities of the wine with a low alcohol content are improved by injecting at least a portion of the dense yeast cream recovered in the settling vat, after having treated said yeasts, either by thermal treatment, or by partial enzymatic digestion, to ensure the breeding of the wine.

The present invention also relates to a fermenting-desugaring unit for implementing the method defined above. Such a fermenter comprises a cylindrical casing and a duct which is arranged substantially coaxially inside the casing and which is held relative to it, the duct being open at both ends, while the cylindrical casing is closed at its lower part, to maintain the aliquot of must to be processed, a controlled heating means being carried by the casing and cooperating with the movement of the circulating flow of the aliquot in the duct, the lower end of the duct comprising an insufflation of air while the upper end of the duct opens freely into the casing.

In a preferred embodiment, the fermenting-desugaring unit according to the invention comprises a controlled heating means, which is advantageously chosen from the group formed by a heating plate forming the lower part of the casing, a heating shell surrounding the lower wall of the casing and the combination of these two types of elements.

In one embodiment of the fermenting-desugaring unit according to the invention, the casing and the duct are coaxial cylinders of substantially vertical axis, the passage section in the duct being substantially equal to the annular passage section between the duct and the casing, so that the speed of displacement of the aliquot of the must being processed on both sides of the duct wall remains substantially constant, the upper level of the aliquot of the must being above the upper end of the duct, the must flowing downwards into the annular passage and upwards in the duct.

In one embodiment, the filling of the fermenting-desugaring unit according to the invention is carried out by means of a pump, which takes, in the wine making vat, an aliquot of must to be processed and transfers it into the fermenting-desugaring unit until the level of the aliquot reaches above the upper end of the duct, this level being marked on the casing by a high-level sensor to allow automatic control of the operation.

In one embodiment of the fermenting-desugaring unit according to the invention, provision is made for emptying the fermenting-desugaring unit into a settling vat by means of a pump, the operation of which is managed by means of a low-level sensor positioned at the same height as a draw-off valve located on the lower part of the fermenting-desugaring unit casing.

In one embodiment of the fermenting-desugaring unit according to the invention, the heating means of the fermenting-desugaring unit is associated with at least one heat probe regulating the temperature of the aliquot of the must being processed for avoid local overheating of the must.

In one embodiment, the fermenting-desugaring unit according to the invention is equipped, in its annular passage, at the median zone of the duct, with a refractometer for the continuous measurement of the sugar content of the circulating aliquot of must.

In one embodiment of the fermenting-desugaring unit according to the invention, the upper part of the casing carries a devesiculation device, which receives the gases, vapors or vesicles emanating from the casing to recover at best the liquid or liquefiable materials that are an integral part of the aliquot. In this embodiment, the wall of the devesiculation device is advantageously maintained on the casing by removable fasteners and comprises a cooling means, for example an outer shell.

In an embodiment where the fermenting-desugaring unit cooperates with a settling vat, the settling vat comprises a cooling means for bringing the aliquot of must be processed to a temperature between 10 and 20° C. for a period of time sufficient to achieve the desired clarification before returning said aliquot to the wine making vat.

To better understand the subject-matter of the invention, an embodiment shown in the accompanying drawing will be described below.

In this drawing:

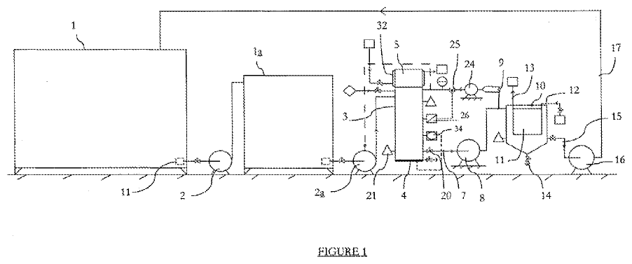

FIG. 1 shows schematically, in elevation, the entirety of an installation for exploiting the method according to the invention;

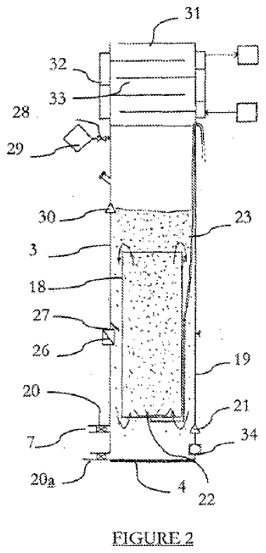

FIG. 2 shows schematically, in axial section, the fermenting-desugaring unit used in the installation of FIG. 1.

Referring to the drawing, we see that 1 designates a wine making vat where the contents of the vat can be taken by a pump 2, the outlet of the vat 1 being equipped with a filter 11. The pump 2 feeds an intermediate vat 1a, which serves to send downstream a predetermined quantity of the wine from the vat 1. The outlet of the intermediate vat 1a is connected to a pump 2a, which feeds the fermenting-desugaring unit designated by 3 as a whole.

The fermenting-desugaring unit 3 has externally a generally cylindrical shape; it is closed at its base by a hot plate 4 and is surmounted by a droplet separator 5, which is attached at the upper part of the fermenting-desugaring unit 3 by removable fasteners. The droplet separator 5 has peripherally a double casing 32 fed with refrigerated water; it internally comprises a series of baffles 33 and it allows to recover substantially the total volume of the processed fraction loaded in the fermenting-desugaring unit 3.

The lower part of the fermenting-desugaring unit 3 is connected by the pipe 7 to a pump 8, which feeds through the pipe 9 a settling vat designated by 10 as a whole. The settling vat 10 constitutes a tank in which there is a cooling element 11, whose cooling fluid, for example water, reaches it through the pipe 12; the outflow of the cooling fluid takes place through the pipe 13. The liquid, which reaches the settling vat 10 through the pipe 9, is cooled by the cooling element 11 and decanted in the tank 10, the decanted product forming a kind of a cream constituted by yeasts that can be evacuated by a valve 14. At the base of the cooling element 11, a pipe 15 is provided with a valve and connected to a pump 16, whose outlet 17 constitutes a return to the top of the wine making vat 1.

The fermenting-desugaring unit 3 is delimited externally by cylindrical casing 19, which is intended to receive the aliquot of must being processed from pump 2a. This cylindrical casing internally contains a cylindrical duct of the same axis, the diameter of which has been defined so that the cross section of the cylindrical duct 18 has a surface substantially equal to that of the cylindrical ring located between the casing 19 and the duct 18. The outer casing of the fermenting-desugaring unit 3 has been designated by 19. A drain pipe 20a fitted with a valve is located at the base of the casing 19; a low-level sensor 21 is located substantially at the level of the pipe 7.

The base of duct 18 comprises an air insufflation device 22; the air supply of the device 22 is provided by a pump 24, whose outlet is equipped with a solenoid valve 25 controlled in particular by the measurements made by a refractometer 26. The tube 23, which goes up along the wall of the casing 19, can distribute the air, which is dispersed at the base of the duct 18 by any suitable means, for example by sintered elements.

The refractometer 26 is carried by the casing 19 and is arranged in the annular space 18/19; it is protected from the liquid stream, which moves in said annular space, by means of an oblique deflector 27, which avoids, on the optics, a deposit of yeasts capable of disturbing the measurement of the sugar content made by the refractometer 26, without an error occurring as a result of bubble formation in the reading zone.

When starting the installation, it is necessary to fill the fermenting-desugaring unit 3 with a pre-calculated quantity of the must contained in the wine making vat 1 and the introduction of this aliquot of must occurs through the valve 28, which is associated with a high-level sensor 29. During operation, the level is monitored by a level sensor 30. When the must has been introduced, heating is started by means of the heating plate 4 and an anti-foam food and nutritional supplements of yeasts are introduced, as is known. This corresponds to the start of the aerobic fermentation phase.

A temperature regulation is implemented by activating the power supply of the heating plate 4 and a regulation of the air flow supplied by the pump 24; it is possible to provide, for example, a nominal air flow rate of approximately 300 L/min. The temperature is preferably set at about 40° C.

It is imperative that yeasts have the necessary amount of O₂, so as not to produce alcohol. Since yeasts are organisms with a high multiplication capacity, their need for air is variable. Therefore, according to the invention, it has been envisaged to use a photometer, which continuously measures the growth of the yeasts and thus allows to use this data to ensure a regulation of the air flow feeding the fermenting-desugaring unit.

As it follows from the indications previously given in this specification, it is essential to stop the air supply just when all the sugar of the fraction being processed is consumed, otherwise an oxidation of the yeasts would occur and an unwanted interference with the taste of wine would take place. This is the reason why, according to the invention, a refractometer 26 was used in the fermenting-desugaring unit 3, the continuous measurements of which have been carefully protected from disturbances to ensure the reliability of the method according to the invention. The set of regulations that can be implemented with the fermenting-desugaring unit according to the invention allows to provide all the functions required by automatisms that can be managed by a PLC.

At the start of the operation of the fermenting-desugaring unit, it is desirable to reach 40° C. as soon as possible; but it is essential not to exceed 60° C., on the one hand to preserve the yeasts and, on the other hand, not to caramelize the sugar of the grape juice on the hotplate 4. It is therefore necessary to check the temperature of the heating element 4 with at least one temperature sensor 34 located in the vicinity of the heating element 4.

The method, which has just been described, allows to treat a wine for which D=10 to 15 alcohol degrees and to obtain a wine for which D-d is between 0.5 and 3 alcohol degrees. It can be used with any commercial active dry yeast (ADY) used in oenology to achieve a desugaring phase. When a settling vat 10 is used, the yeasts, which settle in the settling vat during the clarification phase, represent at least 90% of the yeast population, which allows a high valuation of the method according to the invention.

1. A method for preparation of a wine having good organoleptic qualities and a low alcohol content d, from a must of grapes, whose high sugar content would lead, after a conventional fermentation process, to a wine with a potential alcoholic content D>d, said must being in a wine making vat from which an aliquot of must is taken in a fermentation system using yeasts, said system comprising two fermentation steps, one of which is aerobic and the other anaerobic, the outlet flow of the first step feeding the second step, which provides the processed aliquot and returns it in the wine making vat to be mixed with the remaining initial must, wherein, in a single fermenting-desugaring unit, a simultaneous homogenization of the fermentation medium and aeration of yeasts takes place, the aerobic multiplication of the yeasts consuming sugar without producing ethanol, by means of an air circulation associated with a circulating flow of the aliquot from the wine making vat, said homogenization being achieved with controlled heating, which brings the temperature of the aliquot to a value corresponding to an appropriate fermentation in aerobic mode until a complete or almost complete consumption of the sugar of the aliquot of must being processed, and wherein an aliquot devoid of sugar being processed is transferred from the fermenting-desugaring unit to a settling vat equipped with a cooling means to obtain the desired clarification of the must.

2. The method of claim 1, wherein the circulating flow of the aliquot of must being processed and the associated air flow take place from bottom to top, parallel to the average line of the fermenting-desugaring unit.

3. The method of claim 1, wherein heating the fermenting-desugaring unit maintains, in the fraction of the must being processed that is contained therein, a substantially homogeneous temperature comprised between 30 and 45° C.

4. The method of claim 1, wherein the aliquot of the must devoid of sugar reintroduced in the wine making vat contains yeasts, and comes from the fermenting-desugaring unit, in order to seed the must remaining in said vat to ensure a faster restart of the wine making process.

5. The method of claim 1, wherein in a cleaning or disinfection phase of the fermenting-desugaring unit, the heating of the fermenting-desugaring unit is used to bring to a boil a quantity of wash water which, after condensation, is evacuated through a drain valve.

6. The method of claim 1, wherein the sugar content of the aliquot of must being processed, which circulates in the fermenting-desugaring unit, is continuously measured by means of a refractometer to regulate automatically the aerobic mode of fermentation by activating the air supply.

7. The method of claim 1, wherein the measurement of the sugar level of the aliquot being processed in the fermenting-desugaring unit is used to determine the moment when all the sugar of said fraction has been consumed by the aerobic fermentation and then order the immediate shutdown of the air inflow and heating in the fermenting-desugaring unit, to avoid any deterioration of the organoleptic qualities of said aliquot.

8. The method of claim 1, wherein the microbial density, and thus the growth of the yeasts, is measured in the aliquot being processed by means of a photometer, thus controlling in a complementary manner fermentation in the fermenting-desugaring unit.

9. The method of claim 1, wherein all or part of the yeasts sedimented in the form of a dense yeast-laden cream are collected in the settling vat and reintroduced in the wine making vat to ensure, at less partially, the seeding of said vat.

10. The method of claim 1, wherein the organoleptic qualities of wine with low alcohol content are improved by injecting at least a portion of the dense cream of yeasts recovered in the settling vat, after having processed said yeasts, either by heat treatment or by partial enzymatic digestion to ensure the breeding of the wine.

11. A fermenting-desugaring unit (3) for implementing the method of claim 1, comprising a cylindrical casing (19) and a duct (18), which is arranged substantially coaxially inside the casing (19) and which is held in relation to the casing, the duct (18) being open at both ends, while the cylindrical casing (19) is closed at its lower part, to maintain the aliquot of must to be processed, the lower end of the duct comprising an insufflation of air while the upper part of the duct opens freely into the casing, the section of passage in the duct being substantially equal to the annular passage section between the duct and the casing, so that the speed of displacement of the aliquot of the must being processed on both sides of the wall of the duct remains substantially constant, the upper level of the aliquot of the must being above the upper end of the duct, the must flowing downwards in the annular passage and upwards in the duct, wherein a controlled heating means (4) being carried by the casing (19) and cooperating with the movement of the circulating flow of the aliquot in the duct (18).

12. The unit of claim 11, wherein the controlled heating means is selected from the group consisting of a heating plate (4) forming the lower part of the casing (19), a heating shell surrounding the lower wall of the casing, and a combination of these two types of elements.

13. The unit of claim 11, wherein the heating means (4) is associated with at least one temperature probe (34) regulating the temperature of the aliquot of the must being processed, to avoid local overheating of the must.

14. The unit of claim 11, wherein a refractometer (26), for the continuous measurement of the aliquot of the circulating must, is arranged at the median zone of the duct (18) in the annular passage.

15. The unit of claim 11, wherein the upper part of the casing (19) comprises a devesiculation device (31) comprising a cooling means, which receives the gases, vapors or vesicles emanating from the casing (19) to recover at best the liquid or liquefiable materials that are integral with the aliquot.

16. The unit of claim 11, wherein the settling vat (10) comprises a cooling means (11) for bringing the aliquot of must being processed to a temperature of between 10 and 20° C. for a time sufficient to achieve the desired clarification before returning said aliquot in the wine making vat.
